(19) United States
(12) Patent Application Publication
(10) Pub. No.: US 2006/0111750 A1
(43) Pub. Date: May 25, 2006

(54) Automated external defibrillator (AED) with discrete sensing pulse for use in configuring a therapeutic biphasic waveform

(76) Inventor: Kyle R. Bowers, Boxborough, MA (US)

Correspondence Address: Mark J. Pandiscio;Pandiscio & Pandiscio, P. C., 470 Totten Pond Road, Waltham, MA, 02451-1914, US

(21) Appl. No.: 11/286,870

(22) Filed: Nov. 23, 2005

Related U.S. Application Data
(60) Provisional application No. 60/630,894, filed on Nov. 24, 2004.

(51) Int. Cl. A61N 1/39

(52) U.S. Cl. 607/5

(57) ABSTRACT

An automatic external defibrillator (AED) with a discrete sensing pulse for use in configuring a therapeutic biphasic waveform. The sensing pulse is used to determine a patient-specific parameter (e.g., thoracic impedance) prior to delivery of the therapy waveform. The defibrillator adjusts the therapy waveform, based on the patient-specific parameter, prior to delivery to the patient.

### REFERENCE TO PENDING PRIOR PATENT APPLICATION

This patent application claims benefit of pending prior U.S. Patent Application Ser. No. 60/630,894, filed Nov. 24, 2004 by Kyle R. Bowers for AUTOMATED EXTERNAL DEFIBRILLATOR WITH BIPHASIC WAVEFORM AND DISCRETE SENSING PULSE (Attorney's Docket No. ACCESS-7 PROV), which patent application is hereby incorporated herein by reference.

### FIELD OF THE INVENTION

The present invention relates generally to a defibrillator system and method for producing a discrete sensing pulse for use in configuring a therapeutic biphasic waveform.

### BACKGROUND OF THE INVENTION

Approximately 350,000 deaths occur each year in the United States alone due to sudden cardiac arrest (SCA). Worldwide deaths due to SCA are believed to be at least twice that of the U.S. incidence. Many of these deaths can be prevented if effective defibrillation is administered within 3-5 minutes of the onset of SCA.

SCA is the onset of an abnormal heart rhythm, lack of pulse and absence of breath, leading to a loss of consciousness. If a normal pulse is not restored within a few minutes, death typically occurs. Most often, SCA is due to ventricular fibrillation (VF), which is a chaotic heart rhythm that causes an uncoordinated quivering of the heart muscle. The lack of coordinated heart muscle contractions results in inadequate blood flow to the brain and other organs. Death typically ensues unless this chaotic rhythm is terminated, allowing the heart to restore its own normal rhythm. Defibrillators accomplish this by producing a fast, high-current electrical pulse that, when applied to a patient, momentarily stops the heart, allowing the heart's electrochemical system to recover.

Rapid defibrillation is the only effective means to restore the normal heart rhythm and prevent death after SCA due to ventricular fibrillation. For each minute that passes after the onset of SCA, the rate of mortality generally increases by 10%. If the heart is defibrillated within 1-2 minutes, survival rates can be as high as 90% or more. With delays of approximately 7-10 minutes, the survival rate drops to below 10%. Thus, the only effective solution to VF is early defibrillation.

Automatic External Defibrillators (AEDs) can provide early access to defibrillation, but they must be: (i) easy to use so that they may be administered by a broad range of first responders; (ii) portable so they can be easily carried to an SCA victim; and (iii) easily maintained so as to ensure high reliability. In addition, AEDs must be affordable so that they can be broadly deployed and they must be readily accessible when a SCA event occurs.

AEDs require a portable energy source so as to enable the device to be rapidly deployed to timely treat an SCA victim. Often, the victim may be in a remote or difficult to reach location making compact and portable AEDs most useful to police, emergency medical services (EMS), Search-And-Rescue and other rescue or emergency services.

AEDs must adjust the parameters (e.g., voltage and/or current) of the therapeutic shock which is applied to the patient depending on the specific thoracic impedance of the patient. Thoracic impedances typically vary from patient to patient, thus the defibrillator must either use a sensing pulse to measure the patient's thoracic impedance prior to defibrillation and then adjust the defibrillation voltage prior to delivery of a shock to the patient, or measure the patient's thoracic impedance during defibrillation and then attempt to adjust the therapy waveform during delivery of a shock to the patient.

Some prior art defibrillators measure patient thoracic impedance first, prior to defibrillation, and then charge the defibrillator's capacitors to a predetermined voltage, based on the measured patient thoracic impedance, before delivering the therapeutic waveform to the patient (i.e., a shock capable of defibrillating a patient). However, this approach leads to increased size and complexity of the AED. Other prior art defibrillators adjust the waveform based on patient-specific parameters during the therapy portion of the waveform or during a pre-pulse that is integral to the therapy waveform. As is well known in the art, many defibrillators also attempt to control the “tilt” of the waveform (i.e., the rate at which the capacitors discharge). The disadvantage of this technique is that the control of the tilt must be done during the therapy portion of the waveform, which increases the complexity of the waveform controller.

Older prior art defibrillators use preset voltages and do not control or limit the peak patient current. This technique may generate high peak current for low impedance patients, which may result in myocardial damage.

Thus, there is a need for a new and improved defibrillator system and method for producing a discrete sensing pulse for use in configuring a therapeutic biphasic waveform.

### SUMMARY OF THE INVENTION

The present invention is a defibrillator system and method for producing a discrete sensing pulse for use in configuring a therapeutic biphasic waveform.

More specifically, the sensing pulse is independent of the therapy waveform and is used to determine a patient's thoracic impedance. The sensing pulse uses large signal current levels to accurately measure the patient's thoracic impedance before the therapy waveform is applied. The sensing pulse is short in duration, sufficiently time-separated from the therapy waveform so as to not contribute to the therapy waveform, and does not contain enough energy to itself defibrillate a patient.

In accordance with one aspect of the present invention, the AED has a controller system which contains a microprocessor, memory, an analog-to-digital converter (ADC) and other circuitry to control functionality of the AED.

In accordance with another aspect of the present invention, the AED's controller system contains Flash, RAM and EEPROM memory.

In accordance with another aspect of the present invention, the AED contains a battery pack, high-voltage capacitors, a circuit to charge the capacitors and a circuit to deliver a biphasic waveform and a discrete sensing pulse.

In accordance with another aspect of the present invention, the AED contains a set of pads (i.e., electrodes) that are applied directly to the patient from the defibrillator. These pads comprise an electrically conductive hydrogel that adheres to the patient's skin and provides good electrical connectivity to the patient's chest. The defibrillator produces a voltage potential at the electrodes, which causes a flow of electrical current through the patient's chest.

In accordance with another aspect of the present invention, the defibrillator comprises an LCD display, voice playback circuitry, an audio amplifier and a speaker to guide the user while performing a rescue. Predetermined scripts are played audibly and/or visibly, and instruct the user in the steps of using the AED and providing patient care.

In accordance with another aspect of the present invention, the controller system contains a circuit to sense the current passed through the patient.

In accordance with another aspect of the present invention, the controller system contains a circuit to sense the voltage applied to the patient.

In accordance with another aspect of the present invention, the defibrillation system has current overload protection circuitry that limits the peak current delivered to the patient and protects the defibrillator's high-voltage circuitry.

In accordance with another aspect of the present invention, the defibrillator has a removable flash memory card for logging self-test information and results, and for logging information about the device during a rescue.

In accordance with another aspect of the present invention, the defibrillator stores the patient's electrocardiogram data on the flash memory card for post-incident review of heart rhythms.

In accordance with another aspect of the present invention, the defibrillator has audio recording circuitry and stores the rescue audio data on the flash memory card, which can be played back for post-incident review.

In accordance with another aspect of the present invention, the defibrillator controller stores information about the therapy waveform on the flash memory card.

In accordance with another aspect of the present invention, the defibrillator has power control circuitry that turns the device power on and off in response to signal inputs.

In accordance with another aspect of the present invention, the defibrillator has a real-time clock with an interrupt that enables the power control circuitry to turn on the device.

In accordance with another aspect of the present invention, the defibrillator contains a system monitor circuit that resets the controller system in the event of a microprocessor crash.

In accordance with another aspect of the present invention, the defibrillator contains buttons for controlling the defibrillator.

In accordance with another aspect of the present invention, the AED performs self-tests to ensure proper functionality and device readiness. A status indicator is used to inform the user of device readiness. The status indicator is audible and/or visual, depending on the result of the self-test performed.

In one form of the present invention, there is provided a defibrillator for selectively delivering a therapeutic biphasic waveform to a patient, the defibrillator comprising:

apparatus for applying a discrete sensing pulse to the patient and measuring the return so as to determine a patient-specific parameter prior to delivering the therapeutic biphasic waveform; and

apparatus for applying a therapeutic biphasic waveform to the patient, wherein the therapeutic biphasic waveform is adjusted, according to the measured patient-specific parameter, prior to delivery to the patient.

In another form of the present invention, there is provided a method for selectively delivering a therapeutic biphasic waveform to a patient, the method comprising:

applying a discrete sensing pulse to the patient and measuring the return so as to determine a patient-specific parameter prior to delivering the therapeutic biphasic waveform; and

applying a therapeutic biphasic waveform to the patient, wherein the therapeutic biphasic waveform is adjusted, according to the measured patient-specific parameter, prior to delivery to the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic diagram of the defibrillator and electrodes attached to the patient;

FIG. 2 is a block diagram of the defibrillator components;

FIG. 3A and FIG. 3B are screen displays from an oscilloscope depicting two different configurations of a 360 Joule defibrillator waveform;

FIG. 4 is a graph of the defibrillator sensing pulse current over the impedance range;

FIG. 5 is a table showing an example of the capacitor stacking and therapy waveform parameters for a 200 J therapy waveform; and

FIG. 6 is a table showing an example of the capacitor stacking and therapy waveform parameters for a 360 J therapy waveform.

### DETAILED DESCRIPTION OF THE PREFERRED EMBODIMENTS

The present invention is a defibrillator system and method for producing a discrete sensing pulse for use in configuring a therapeutic biphasic waveform.

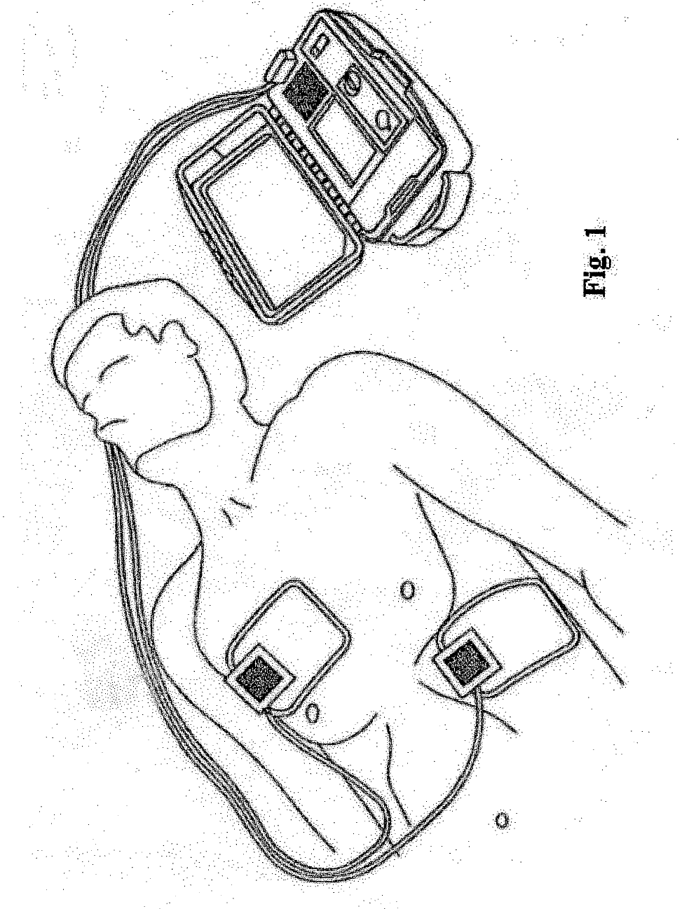

As shown in FIG. 1, the patient is connected to the AED via a pair of electrodes, which are attached directly to the skin of the patient's chest. The defibrillator uses the electrodes to provide defibrillation shocks to the patient, where a pulsed electrical current is passed through the patient's heart. The AED also uses the electrodes to first sense ECG signals from the patient so as to determine the condition of the patient's heart (i.e., shockable or not). The electrodes contain a conductive hydogel, which secures the pads to the patient's skin and provides good electrical conductivity. The electrodes are terminated with a connector, which is generally connected to the defibrillator after the pads have been applied to the patient.

In a preferred embodiment of the present invention, the electrodes are sealed in a tray, which resides in the lid of the AED unit. The electrodes are discarded after use and the tray is replaced.

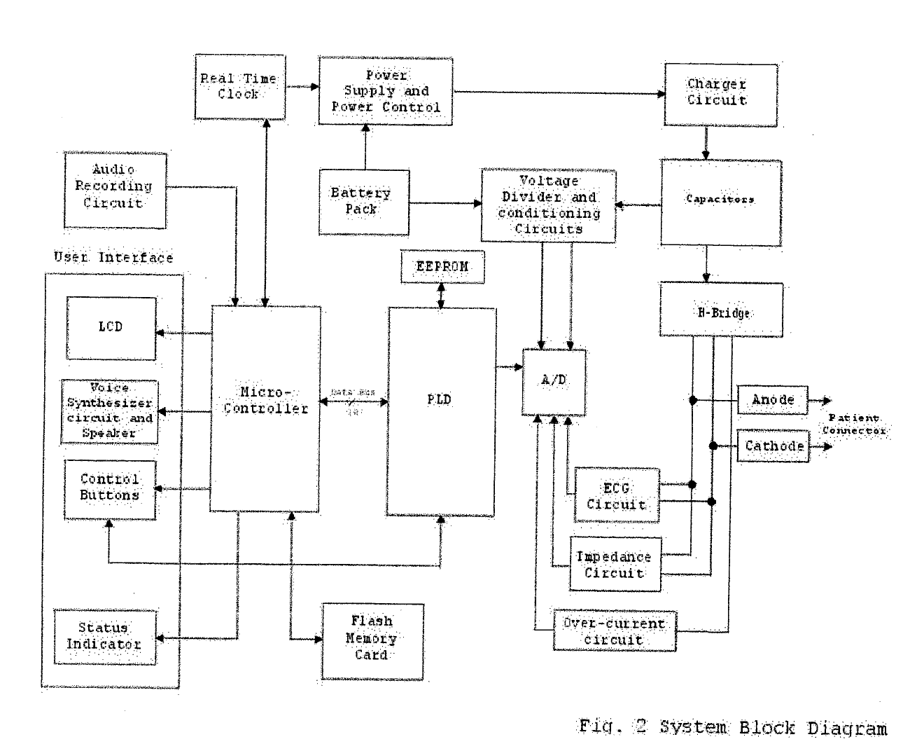

Looking now at FIG. 2, there is shown a block diagram of the AED components. The AED contains a controller system including, but not limited to, a microprocessor (MicroController), programmable logic device (PLD), memory and an analog-to-digital converter (ADC). In one preferred embodiment of the invention, the microprocessor executes instructions to: (i) sample the data; (ii) store the data into memory; and (iii) process data. In the preferred embodiment, the programmable logic device (PLD) controls the interface to the analog-to-digital converter (ADC) and stores the sampled data into a local memory buffer. The programmable logic device (PLD) then interrupts the microprocessor to sample the data contained in the buffer, via a data bus connected between the microprocessor and the PLD. The microprocessor may also directly interface to the analog-to-digital converter (ADC) and use internal timing or interrupts for sampling data. Additionally, the microprocessor may be a microcontroller and have the memory, analog-to-digital converter (ADC) and other peripherals on a single chip.

The analog-to-digital converter (ADC) is connected to circuits which measure the patient's electrocardiogram (ECG), the patient's transthoracic impedance, the AED temperature, the AED's capacitor charger circuits, the current passed through the patient, the voltage applied to the patient and other analog circuits.

The AED also contains the conventional electrical components used to generate defibrillation shocks including, but not limited to, a battery pack, capacitor charger circuit, high-voltage capacitors and an H-bridge circuit.

In a preferred embodiment of the present invention, the PLD controls: (i) the charger circuit (ii) the charging of the capacitors to a target voltage level; (iii) charge refreshing; and (iv) hysteresis.

In a preferred embodiment of the present invention, the defibrillator uses a capacitor stacking circuit technique to control the voltage level (and hence the current) delivered to the patient by the AED, based on the prior determination of the patient's transthoracic impedance.

In another preferred embodiment of the present invention, the PLD controls the waveform delivery system including, but not limited to, the H-Bridge circuit and the capacitor stacking circuit.

In a preferred embodiment of the present invention, the defibrillator contains a removable flash memory card. The defibrillator uses the flash memory card to store pertinent data. Examples of such data include, but are not limited to, a patient's ECG data, a patient's transthoracic impedance, the defibrillator's self-test results, environment data, device use data, diagnostic information, therapy waveform data and other relevant device data.

In a preferred embodiment of the present invention, the flash memory card is a multi-media card. In other preferred embodiments, the flash memory card may be CompactFlash, synchronous digital or similar flash card types.

The defibrillator also contains an LCD screen, voice synthesizer and speaker for instructing the rescuer during device use. The voice synthesizer and speaker are also capable of producing tones. These components are also used for the status indicator system. The LCD screen and tones are used to notify the user of the self-test result, a potential user action to take and an error code if a critical self-test has failed. An example of a potential user action is to replace a depleted battery before attempting to defibrillate a patient. Another example of a user action is to replace out-of-date pads, before placing the device back in to service.

The defibrillator also contains a number of buttons for user control. These buttons include, but are not limited to, a power button, a shock button and one or more special purpose buttons. A preferred embodiment of the present invention includes buttons to manually control the defibrillator.

The defibrillator also contains an audio recording circuit that is used to record rescuer's voices and other audible events. The audio recording circuit contains a small microphone and a digital recording integrated circuit (IC), which compresses and buffers the audio data. The controller system reads the data from the recording IC's buffer and stores the data on the removable flash card.

Traditional defibrillators adjust the therapy waveform based on patient-specific parameters during the therapy portion of the waveform or during a pre-pulse that is integral to the therapy waveform (i.e., the pre-pulse contributes to the therapy waveform). Many defibrillators additionally attempt to control the “tilt” of the waveform (i.e., the rate at which the capacitors discharge), during delivery of the therapeutic waveform.

In a preferred embodiment of the present invention, the sensing pulse is independent of the therapy waveform (i.e., the sensing pulse does not contribute to the therapy waveform). The patient dependent parameters are measured during the sensing pulse and decisions about the therapy waveform are made before the therapy waveform is delivered.

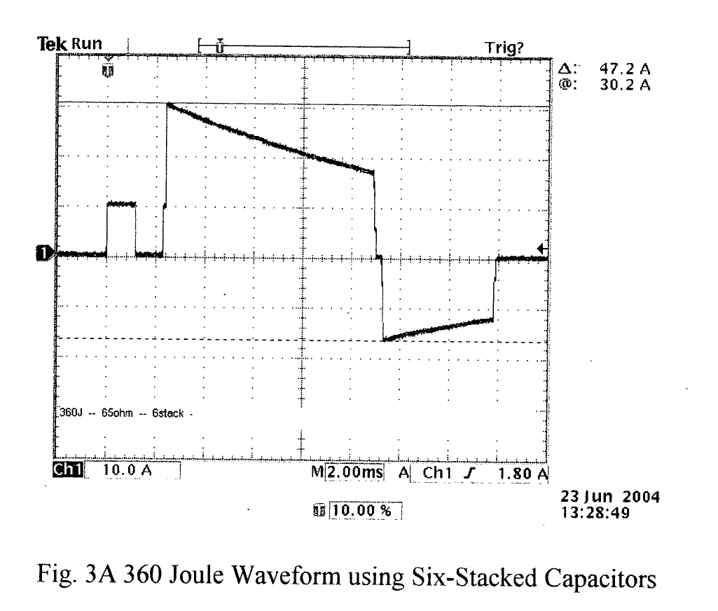

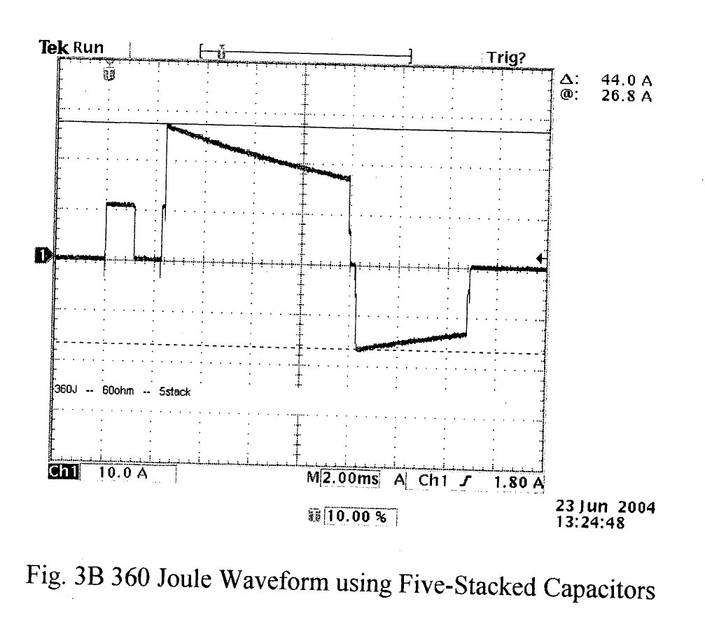

In a preferred embodiment of the present invention, the sensing pulse is used to determine a patient's transthoracic impedance. The sensing pulse uses large signal current levels to accurately determine this parameter before the therapy waveform is applied. The sensing pulse is short in duration, sufficiently time-separated from the therapy waveform so as to not contribute to the therapy waveform, and does not itself contain enough energy to defibrillate a patient. FIGS. 3A and 3B is an illustration of a sensing pulse and therapeutic waveform generated, in accordance with the present invention. The sensing pulse does not significantly discharge the high-voltage capacitors, thereby leaving the capacitors substantially fully charged.

In a preferred embodiment of the present invention, the duration of the sensing pulse is one millisecond. However, the sensing pulse could be much shorter in duration. As those skilled in the art can appreciate, the sensing pulse duration only needs to be long enough for the controller to take a sample of the current passed through the patient and/or the voltage once it is at steady-state.

In a preferred embodiment of the present invention, the controller uses a single sample of the return of the sensing pulse to determine patient impedance and hence the appropriate therapy waveform parameters. In another preferred embodiment of the present invention, the controller uses several samples of the return of the sensing pulse to produce an average result which is then used to determine patient impedance and hence the appropriate therapy waveform parameters.

In a preferred embodiment of the present invention, the sensing pulse is at least one-millisecond apart from the therapy waveform. In other aspects of the present invention the sensing pulse could be further apart from the therapy waveform. Additionally, in other aspects of the present invention, the sensing pulse could be to some extent closer to the therapy waveform. It will be appreciated that the voltage of the sensing pulse and the time duration of the sensing pulse together determine the time interval between the sensing pulse and the therapy waveform which is necessary to differentiate the sensing pulse from the therapy waveform.

In a preferred embodiment of the present invention, the defibrillator may optionally not deliver a therapy waveform. As those skilled in the art can appreciate, the controller may not deliver therapy to a patient due to the results of the sensing pulse because: (i) the sensing pulse current is too high, which could indicate an over-current (pad shorting) condition; or (ii) the sensing pulse current is too low, which could indicate an open circuit possibly due to pad detachment from the patient.

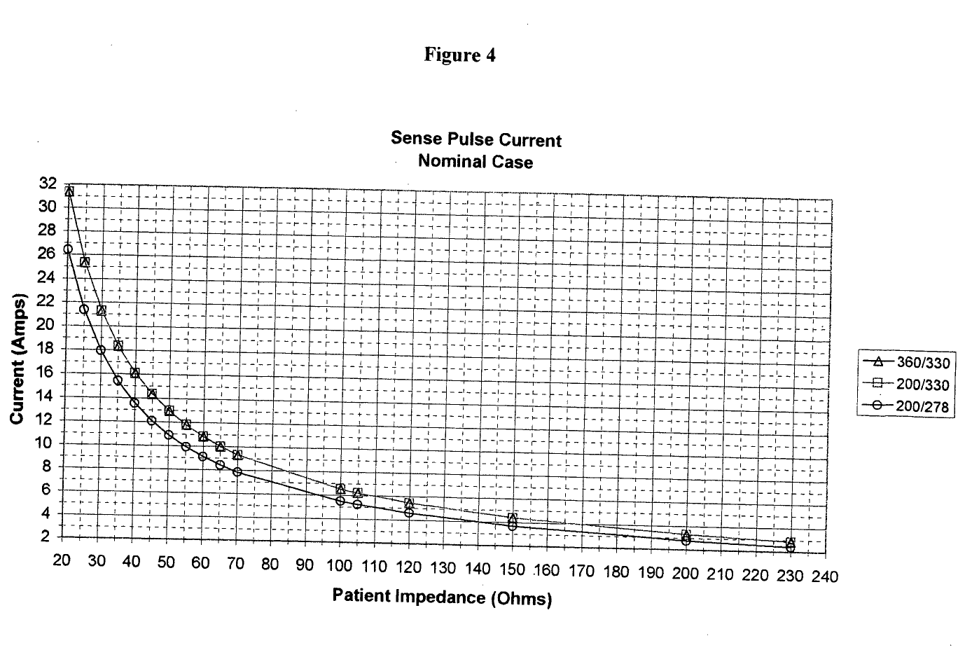

The sensing pulse current is shown in the graph of FIG. 4. The current is plotted over the patient impedance range of the defibrillator. As is well known in the art, the typical impedance range of patients is 60 to 100 ohms.

In a preferred embodiment of the present invention, the defibrillator uses six high-voltage capacitors which may be stacked. The higher the patient's impedance, the more capacitors that are stacked. As is well known in the art, the defibrillator uses switches to configure the capacitors in series and/or parallel so as to obtain the desired “firing” configuration.

The defibrillator may use one or more capacitors stacked to deliver the sensing pulse. In a preferred embodiment of the present invention, the defibrillator uses a two-capacitor stack to deliver the sensing pulse.

The defibrillator may use one or more capacitors stacked to deliver the therapy pulse. In addition, the defibrillator may use an array of small capacitors arranged in multiple series (stacked) and parallel configurations to deliver the therapy pulse.

In a preferred embodiment of the present invention, the defibrillator uses two to six capacitors stacked to deliver the therapy pulse. It will be appreciated that the capacitors are arranged in series and/or parallel so as to obtain the correct amount of defibrillator voltage to be used during the therapy pulse based upon the information received about the patient's impedance from the sensing pulse.

The sensing pulse is used to determine the parameters of variable energy therapy waveforms ranging from 1 Joule to 360 Joules. In a preferred embodiment of the present invention, the defibrillator controller uses the sensing pulse reading to determine the parameters of a 200 J (Joule) therapy waveform. In another preferred embodiment of the present invention, the defibrillator controller uses the sensing pulse to determine the parameters of a 360 J therapy waveform.

In one preferred form of the invention, the defibrillator controller adjusts the timing for the therapy waveform, depending on the reading of the sensing pulse.

In a preferred embodiment of the present invention, the defibrillator controller uses the results of the sensing pulse to determine a variable time for the forward phase of the therapy waveform. As is well known in the art, it is generally advantageous to extend the duration of the forward phase for higher impedance patients.

In a preferred embodiment of the present invention, the defibrillator controller uses a time of 7.5 mS for the forward phase of the therapy waveform for impedances from 20 to 63 ohms and 8.5 mS for impedances from 64 to 200 ohms.

In a preferred embodiment of the present invention, the defibrillator controller uses a fixed time for the reverse phase of the therapy waveform.

In a preferred embodiment of the present invention, the defibrillator controller uses a fixed time of 4.5 mS for the reverse phase of the therapy waveform.

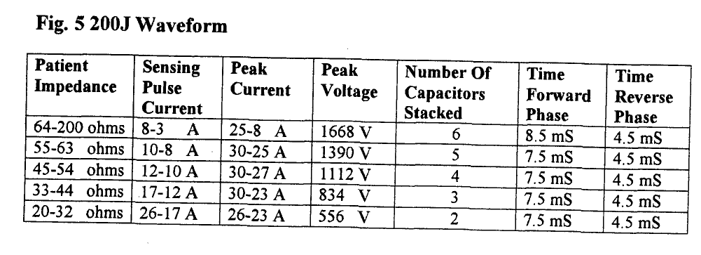

An example of the 200 J therapy waveform parameters is shown in FIG. 5. In this example, each capacitor is charged to 278V. It should be noted that the peak current is limited over the full range of patient impedances.

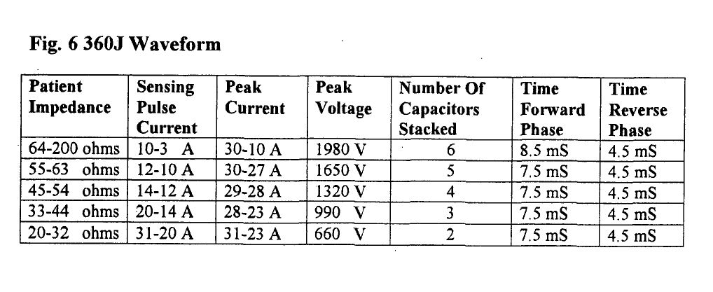

An example of the 360 J therapy waveform parameters is shown in FIG. 6. In this example, each capacitor is charged to 330V. The peak current is also limited in this example. Looking back at FIG. 3A, there is shown a 360 J therapy waveform at a patient impedance of 60 ohms. It can be seen in this example that the sensing pulse is generated by two stacked capacitors and the therapy waveform is generated by six stacked capacitors. FIG. 3B shows a 360 J therapy waveform generated using five-stacked capacitors.

### Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A defibrillator for selectively delivering a therapeutic biphasic waveform to a patient, the defibrillator comprising:
apparatus for applying a discrete sensing pulse to the patient and measuring the return so as to determine a patient-specific parameter prior to delivering the therapeutic biphasic waveform; and
apparatus for applying a therapeutic biphasic waveform to the patient, wherein the therapeutic biphasic waveform is adjusted, according to the measured patient-specific parameter, prior to delivery to the patient.

2. A defibrillator according to claim 1 wherein the measured patient-specific parameter is the patient's transthoracic impedance.

3. A defibrillator according to claim 1 wherein the voltage of the therapeutic biphasic waveform is adjusted according to the measured patient-specific parameter.

4. A defibrillator according to claim 1 wherein the timing of the therapeutic biphasic waveform is adjusted according to the measured patient-specific parameter.

5. A defibrillator according to claim 1 wherein the peak current of the therapeutic biphasic waveform is limited according to the measured patient-specific parameter.

6. A defibrillator according to claim 1 wherein the therapeutic biphasic waveform delivers between 1 and 360 joules to the patient.

7. A defibrillator according to claim 1 wherein the discrete sensing pulse has a duration of between approximately 1 microsecond and 1 millisecond.

8. A defibrillator according to claim 2 wherein the therapeutic biphasic waveform is adjusted for a patient impedance range of 20 to 200 ohms.

9. A defibrillator according to claim 2 wherein the measured patient impedance is determined to be out of range and the therapeutic biphasic waveform is not delivered.

10. A defibrillator according to claim 4 wherein the timing of the therapeutic biphasic waveform is adjusted in the forward phase of the therapeutic biphasic waveform.

11. A method for selectively delivering a therapeutic biphasic waveform to a patient, the method comprising:
applying a discrete sensing pulse to the patient and measuring the return so as to determine a patient-specific parameter prior to delivering the therapeutic biphasic waveform; and
applying a therapeutic biphasic waveform to the patient, wherein the therapeutic biphasic waveform is adjusted, according to the measured patient-specific parameter, prior to delivery to the patient.

12. A method according to claim 11 wherein the measured patient-specific parameter is the patient's transthoracic impedance.

13. A method according to claim 11 wherein the voltage of the therapeutic biphasic waveform is adjusted according to the measured patient-specific parameter.

14. A method according to claim 11 wherein the timing of the therapeutic biphasic waveform is adjusted according to the measured patient-specific parameter.

15. A method according to claim 11 wherein the peak current of the therapeutic biphasic waveform is limited according to the measured patient-specific parameter.

16. A method according to claim 11 wherein the therapeutic biphasic waveform delivers between 1 and 360 joules to the patient.

17. A method according to claim 11 wherein the discrete sensing pulse has a duration of between approximately 1 microsecond and 1 millisecond.

18. A method according to claim 12 wherein the therapeutic biphasic waveform is adjusted for a patient impedance range of 20 to 200 ohms.

19. A method according to claim 12 wherein the measured patient impedance is determined to be out of range and the therapeutic biphasic waveform is not delivered.

20. A method according to claim 14 wherein the timing of the therapeutic biphasic waveform is adjusted in the forward phase of the therapeutic biphasic waveform.
